(19) United States
(12) Patent Application Publication
(10) Pub. No.: US 2024/0112805 A1
(43) Pub. Date: Apr. 4, 2024

(54) PHYSICAL CONDITION DETECTION METHOD, PHYSICAL CONDITION DETECTION DEVICE, AND RECORDING MEDIUM

(71) Applicant: Panasonic Holdings Corporation, Osaka (JP)

(72) Inventors: Maho SHIOTANI, Osaka (JP); Miwa TAKEWA, Osaka (JP); Katsuhisa YAMAGUCHI, Osaka (JP)

(21) Appl. No.: 18/533,760

(22) Filed: Dec. 8, 2023

Related U.S. Application Data
(63) Continuation of application No. PCT/JP2022/022392, filed on Jun. 1, 2022.
(60) Provisional application No. 63/210,261, filed on Jun. 14, 2021.

(51) Int. Cl. G16H 50/20; A61B 5/0205; A61B 5/024; A61B 5/08; G16H 50/30

(52) U.S. Cl. CPC G16H 50/20; A61B 5/0205; A61B 5/024; A61B 5/0816; G16H 50/30

(57) ABSTRACT

A physical condition detection method performed by a computer includes: obtaining activity data including a respiratory rate and a heart rate of a subject during a predetermined time period; calculating a plurality of features, based on the activity data obtained; obtaining an anomaly score indicating a degree of an anomaly in a physical condition per the predetermined time period, by inputting the plurality of features calculated into a model that has learned normality or anomaly in an activity data group; calculating a graded score for indicating a physical condition anomaly level of the subject in a graded manner, based on the anomaly score obtained; and outputting the graded score calculated.

### CROSS REFERENCE TO RELATED APPLICATIONS

This is a continuation application of PCT International Application No. PCT/JP2022/022392 filed on Jun. 1, 2022, designating the United States of America, which is based on and claims priority of U.S. Provisional Patent Application No. 63/210,261 filed on Jun. 14, 2021. The entire disclosures of the above-identified applications, including the specifications, drawings and claims are incorporated herein by reference in their entirety.

### FIELD

The present disclosure relates to a physical condition detection method, a physical condition detection device, and a recording medium.

### BACKGROUND

The 2025 problem in Japan is an aging society problem in which all eight million people belonging to what is called “Dankai no Sedai” (the baby boomer generation) will reach the ages of 75 or older, resulting in a quarter of the nation's population reaching the ages of 75 or older. This problem involves a problem of a labor shortage caused by increasing demands for medical and caregiving services.

Against this backdrop, the number of subjects of nursing and care who are looked after by health care workers and caregivers is increasing. As a result, a small change in a physical condition that may lead to an anomaly in health may be overlooked. If a small change in a physical condition is overlooked, there is a risk that a subject may become more severely ill.

To deal with this, for example, Patent Literature (PTL) 1 discloses a technique of notifying an appropriate recipient of an anomaly in a monitored person when an anomaly in the monitored person is determined. Accordingly, an anomaly of the monitored person can be notified to an appropriate monitoring person in accordance with an anomaly state of the monitored person.

### CITATION LIST

### Patent Literature

PTL 1: WO 2018/116830

### SUMMARY

### Technical Problem

However, PTL 1 described above only discloses a technique of providing the notification in the case where vital information of the monitored person obtained from a sensor indicates an anomalous value, and thus the technique is not capable of detecting a small change in a physical condition that may lead to an anomaly in health of the monitored person, that is, a sign of the anomaly in health.

The present disclosure is made in view of the circumstances described above, and an object of the present disclosure is to provide a physical condition detection method and the like capable of detecting a sign of an anomaly in health of a subject.

### Solution to Problem

A physical condition detection method according to an aspect of the present disclosure is a physical condition detection method performed by a computer and includes: obtaining activity data including a respiratory rate and a heart rate of a subject during a predetermined time period; calculating a plurality of features, based on the activity data obtained; obtaining an anomaly score indicating a degree of an anomaly in a physical condition per the predetermined time period, by inputting the plurality of features calculated into a model that has learned normality or anomaly in an activity data group including a plurality of features; calculating a graded score for indicating a physical condition anomaly level of the subject in a graded manner, based on the anomaly score obtained; and outputting the graded score calculated.

Note these general or specific aspects may be implemented using a system, a device, a method, an integrated circuit, a computer program, or a computer-readable recording medium such as a compact disk read-only memory (CD-ROM), or any combination of systems, devices, methods, integrated circuits, computer programs, or recording media.

### Advantageous Effects

According to the physical condition detection method and the like according to the present disclosure, it is possible to detect a sign of an anomaly in health of a subject, that is, a small change in a physical condition that may lead to the anomaly in health of the subject.

### BRIEF DESCRIPTION OF DRAWINGS

These and other advantages and features will become apparent from the following description thereof taken in conjunction with the accompanying Drawings, by way of non-limiting examples of embodiments disclosed herein.

FIG. 1 is a diagram illustrating an example of a configuration of a physical condition detection system according to an embodiment.

FIG. 2 is a block diagram illustrating an example of a specific configuration of an information management server according to the embodiment.

FIG. 3 is a diagram illustrating an example of distributions of an anomaly data group and a normal data group in an activity data group according to the embodiment.

FIG. 4 is a diagram schematically illustrating a model according to the embodiment.

FIG. 5 is a diagram showing an example of graded scores in five levels and conditions for the graded scores according to the embodiment.

FIG. 6 is a diagram illustrating an example of a display for handing an anomaly in a physical condition of a subject according to the embodiment.

FIG. 7 is a diagram illustrating an example of a display for handing an anomaly in a physical condition of a subject according to the embodiment.

FIG. 8 is a diagram illustrating an example of a display for handing an anomaly in a physical condition of a subject according to the embodiment.

FIG. 9 is a flowchart illustrating an outline of operation of an information management server according to the embodiment.

FIG. 10 is a flowchart illustrating an operation example of the information management server according to the embodiment.

FIG. 11A is a diagram for schematically describing anomaly detection in the case of long-term operation using a model according to a comparative example.

FIG. 11B is a diagram for schematically describing anomaly detection in the case of long-term operation using the model according to the comparative example.

FIG. 12 is a diagram for schematically describing anomaly detection in the case of long-term operation using a model according to the embodiment.

FIG. 13 is a graph schematically illustrating improvement in performance by updating the model according to the present embodiment.

FIG. 14 is a diagram illustrating an example of a linkage display displayed by a display terminal according to Working Example 1.

FIG. 15 is a diagram illustrating incident finding case 1 according to Working Example 2.

FIG. 16 is a diagram illustrating incident finding case 2 according to Working Example 2.

FIG. 17 is a diagram illustrating incident finding case 3 according to Working Example 2.

### DESCRIPTION OF EMBODIMENT

A physical condition detection method according to an aspect of the present disclosure is a physical condition detection method performed by a computer and includes: obtaining activity data including a respiratory rate and a heart rate of a subject during a predetermined time period; calculating a plurality of features, based on the activity data obtained; obtaining an anomaly score indicating a degree of an anomaly in a physical condition per the predetermined time period, by inputting the plurality of features calculated into a model that has learned normality or anomaly in an activity data group including a plurality of features; calculating a graded score for indicating a physical condition anomaly level of the subject in a graded manner, based on the anomaly score obtained; and outputting the graded score calculated.

According to this aspect, it is possible to detect a sign of an anomaly in health of a subject, that is, a small change in the physical condition that may lead to an anomaly in health of the subject.

More specifically, a plurality of features are calculated based on the activity data, and the plurality of features calculated are input into a model that has learned normality or anomaly in an activity data group. Based on the anomaly score obtained as a result of inputting the plurality of features into the model, a graded score indicating evaluation of a physical condition anomaly level of the subject in a graded manner is calculated.

Accordingly, it is possible to become aware of (detect) a sign of an anomaly in health of the subject from the graded score.

Therefore, it is possible to detect a sign of an anomaly in health of the subject that even a health care worker providing care or nursing to the subject fails to find.

Furthermore, since the graded score makes it easy to grasp a degree of the anomaly on-site, handling of various small changes in the physical condition of the subject, that is, various signs of an anomaly in health that may lead to an anomaly in health in daily life of the subject, is facilitated.

Further, for example, the calculating of the graded score may include performing factor analysis when the graded score is greater than or equal to a predetermined value to analyze, for each of elements included in the activity data, whether the element is a factor for the graded score being greater than or equal to the predetermined value, and the outputting of the graded score may include outputting the graded score and an element which has been analyzed to be the factor by the factor analysis.

In this manner, when the graded score is greater than or equal to the predetermined value indicating the need of handling a sign of an anomaly in health, whether a factor for the small change in the physical condition leading to the anomaly in health lies in, for example, the heart rate or the respiratory rate, and the like are notified together.

Therefore, a health care worker who provides care or nursing to the subject can handle the sign of the anomaly in health early and appropriately with the notified factor as a clue.

Here, for example, the activity data may include at least the respiratory rate and the heart rate among food intake, the respiratory rate, the heart rate, and an out-of-bed rate of the subject during the predetermined time period, the out-of-bed rate being a rate at which the subject is out of bed.

As described above, the activity data is obtained on-site day-by-day and includes at least the respiratory rate and the heart rate among food intake, the respiratory rate, the heart rate, and an out-of-bed rate of the subject. Accordingly, the plurality of features are calculated from activity data items obtained on-site day-by-day, and thus a sign of an anomaly in health can be detected with higher precision.

Further, for example, in the calculating of the plurality of features, among a mean value, a maximum value, a standard deviation, a skewness, a kurtosis, and an impulse factor of each of the respiratory rate, difference data on the respiratory rate, the heart rate, and difference data on the heart rate, at least the mean value and the maximum value of each of the respiratory rate and the heart rate may be calculated as the plurality of features, the impulse factor being obtained by subtracting the mean value from the maximum value.

In this manner, the plurality of features are calculated by performing statistical processing and the like on the activity data. Accordingly, from the plurality of features calculated, an anomaly score can be obtained with higher precision using a trained model.

Further, for example, the model may be a model that has learned normality or anomaly in the activity data group through unsupervised learning using the activity data group.

Accordingly, by performing the unsupervised learning on the model using the activity data items obtained on-site day-by-day, it is possible to obtain a trained model that is capable of detecting a sign of an anomaly in health. Accordingly, when the trained model capable of detecting a sign of an anomaly in health is obtained using the activity data of the subject, the model is obtained without a burden on an on-site staff member such as a health care worker of the subject.

Further, for example, the model may be a model that separates an outlier, based on a decision tree.

The activity data group has such a nature that anomaly data less frequently occurs than normal data, and that the anomaly data is different from the normal data in distribution position. By using this nature, a model that separates an outlier based on a decision tree can be used as a model that detects a sign of an anomaly in health from activity data of a subject.

Here, for example, the model may be an isolation forest model.

Further, for example, the model may be regularly updated using the activity data obtained.

Accordingly, the model can be repeatedly updated with activity data being the activity data accumulated after the creation of the model and added to the activity data used in the creation of the model. That is, the model is capable of detecting a sign of an anomaly in health while dealing with fluctuations due to a medium- to long-term disease of the subject or medium- to long-term fluctuations due to environmental influences.

Further, for example, the outputting of the graded score may include: outputting the graded score calculated to a terminal possessed by a monitoring person who monitors the subject; and causing a user interface of the terminal to present a display for the monitoring person to handle an anomaly in a physical condition of the subject.

Accordingly, since the display to handle the anomaly in the physical condition of the subject is presented, an on-site staff member can easily grasp the anomaly in the physical condition of the subject, thus making it easy to handle the anomaly in the physical condition of the subject appropriately. In short, it becomes easier for the on-site staff member to handle various small changes in the physical condition of the subject, that is, various signs of an anomaly in health that may lead to an anomaly in health in daily life of the subject.

A physical condition detection device according to an aspect of the present disclosure includes: a transceiver that obtains activity data including a respiratory rate and a heart rate of a subject during a predetermined time period; a feature calculator that calculates a plurality of features, based on the activity data obtained; an anomaly score calculator that obtains an anomaly score indicating a degree of an anomaly in a physical condition per the predetermined time period, by inputting the plurality of features calculated into a model that is created by a model creator and has learned normality or anomaly in an activity data group including a plurality of features; and a graded score calculator that calculates a graded score for indicating a physical condition anomaly level of the subject in a graded manner, based on the anomaly score obtained, wherein the transceiver outputs the graded score calculated.

A recording medium according to an aspect of the present disclosure is a non-transitory computer-readable recording medium having recorded thereon a program for causing a computer to execute: obtaining activity data including a respiratory rate and a heart rate of a subject during a predetermined time period; calculating a plurality of features, based on the activity data obtained; obtaining an anomaly score indicating a degree of an anomaly in a physical condition per the predetermined time period, by inputting the plurality of features calculated into a model (model creator) that has learned normality or anomaly in an activity data group including a plurality of features; calculating a graded score for indicating a physical condition anomaly level of the subject in a graded manner, based on the anomaly score obtained; and outputting the graded score calculated.

Note these general or specific aspects may be implemented using a system, a device, a method, an integrated circuit, a computer program, or a computer-readable recording medium such as a CD-ROM, or any combination of systems, devices, methods, integrated circuits, computer programs, or recording media.

Hereinafter, an exemplary embodiment of the present disclosure will be described with reference to the drawings. The embodiment described below illustrates a specific example of the present disclosure. The numerical values, shapes, constituent elements, steps, the processing order of the steps etc. illustrated in the embodiment below are mere examples, and are not intended to limit the present disclosure. Among the constituent elements in the embodiment below, those not recited in any of the independent claims will be described as optional constituent elements. Furthermore, in all embodiments, the details can be combined.

### EMBODIMENT

Hereinafter, a physical condition detection method and so on according to the present embodiment will be described with reference to the drawings.

### [1 Physical Condition Detection System 100]

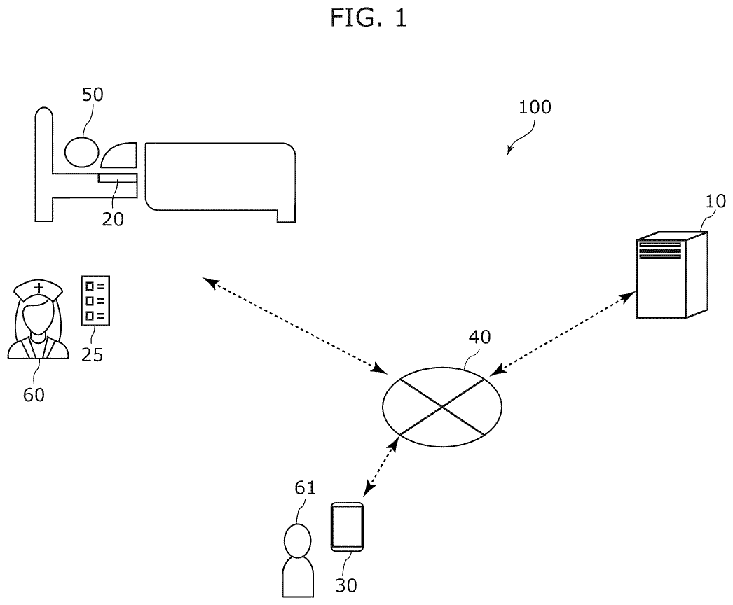

FIG. 1 is a diagram illustrating an example of a configuration of physical condition detection system 100 according to the present embodiment.

Physical condition detection system 100 according to the present embodiment is a system configured such that information management server 10 detects a small change in a physical condition that may lead to an anomaly in health (i.e., a sign of the anomaly in health) of a subject of nursing or care.

As illustrated in FIG. 1, physical condition detection system 100 includes information management server 10, sensor 20, and display terminal 30. These are connected via communication network 40. Communication network 40 may be a wired network, may be a wireless network, or may be both a wired network and a wireless network. FIG. 1 also illustrates subject 50 of nursing or care, user 60 who is an on-site staff member such as a health care worker who provides nursing or care to subject 50, user 61 who can check display terminal 30 and is an on-site staff member such as a monitoring person who monitors subject 50, and recorded data 25 that includes recorded details of nursing or care of subject 50 by user 60. On recorded data 25, for example, food intakes that are amounts of food taken by subject 50 in the morning, afternoon, evening, and the like, and that are input by user 60 being an on-site staff member are recorded.

FIG. 1 illustrates an example of the case where physical condition detection system 100 includes one sensor 20. However, this is not limiting. Sensors 20 as many as the number of subjects 50 of nursing or care may be included.

### [1.1 Sensor 20]

Sensor 20 obtains, by sensing, activity data including respiratory rates and heart rates of subject 50 in a predetermined time period. In the present embodiment, sensor 20 obtains data including a heart rate, a respiratory rate, a body motion, and the like while subject 50 is in bed (hereinafter, will be also referred to as sensor data) every second.

Note that an interval at which the sensor data including the heart rate, the respiratory rate, the body motion, and the like is obtained is not limited to one second. The interval may be two seconds. The interval may be an interval in any unit that enables sensing of changes in the sensor data of subject 50. Sensor 20 may sense whether subject 50 is in bed according to whether sensor 20 can sense the heart rate, the respiratory rate, the body motion, and the like. Sensor 20 may further sense a life rhythm such as a sleep state.

For example, sensor 20 may be a sensor device having a pressure sensor or the like and may be placed in a bed to sense subject 50 every second. In this case, for example, sensor 20 may output, every second, value 1 indicating being out of bed as sensor data indicating that subject 50 is out of bed. For example, sensor 20 may output a sensor data value such as the respiratory rate of subject 50 every second.

### [1.2 Information Management Server 10]

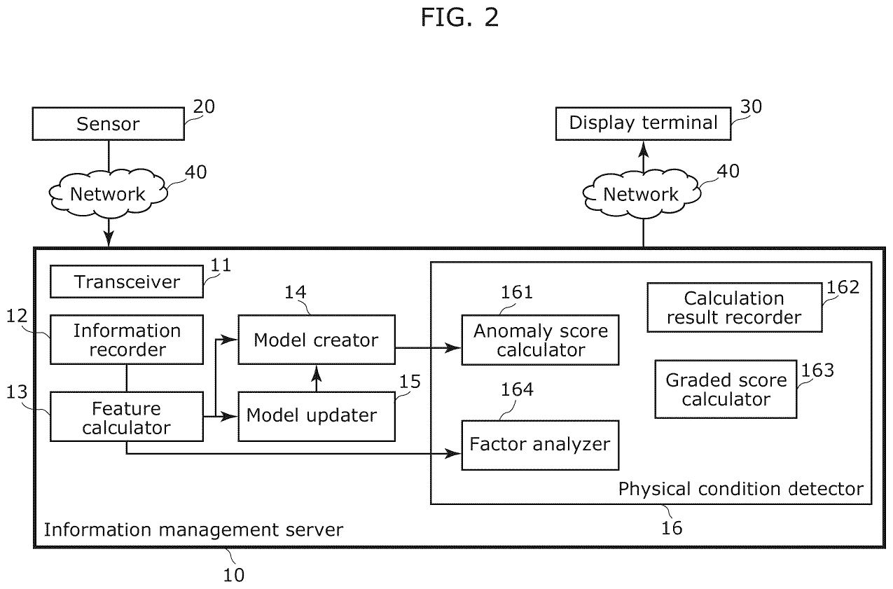

FIG. 2 is a block diagram illustrating an example of a specific configuration of information management server 10 according to the present embodiment.

Information management server 10 is implemented using a computer including, for example, a processor (a microprocessor), memory, a communication interface, and the like. Information management server 10 may operate with a part of the configuration of information management server 10 included in a cloud server. Information management server 10 is an example of a physical condition detection device and detects a small change in a physical condition that may lead to an anomaly in health (i.e., a sign of the anomaly in health) of subject 50.

In the present embodiment, as illustrated in FIG. 2, information management server 10 includes transceiver 11, information recorder 12, feature calculator 13, model creator 14, model updater 15, and physical condition detector 16.

### [1.2.1 Transceiver 11]

Transceiver 11 includes, for example, a communication interface and transmits and receives various types of information to and from sensor 20 or display terminal 30 via communication network 40. Transceiver 11, for example, obtains activity data including the respiratory rate and the heart rate of subject 50 during a predetermined time period. Here, the activity data includes at least the respiratory rate and the heart rate among food intake, the respiratory rate, the heart rate, and an out-of-bed rate of subject 50 during the predetermined time period as described above. Here, the out-of-bed rate is a rate at which subject 50 is out of bed. Further, transceiver 11 outputs the graded score calculated by physical condition detector 16 to a terminal possessed by user 61 such as a monitoring person who monitors subject 50.

In the present embodiment, transceiver 11 obtains the sensor data such as the heart rate, the respiratory rate, and the body motion per second while subject 50 is in bed, from sensor 20 via communication network 40 at predetermined intervals, for example, every minute. Transceiver 11 also obtains, for example, recorded data 25 that includes recorded details of nursing or care for subject 50 by user 60 being an on-site staff member as illustrated in FIG. 1. In this manner, transceiver 11 obtains activity data that includes the sensor data and recorded data 25 and is obtained on-site every day, via communication network 40. Transceiver 11 also transmits a graded score calculated by physical condition detector 16 to display terminal 30 via communication network 40. Note that transceiver 11 may transmit information for a display that is provided on a user interface of display terminal 30 such as a graded score display, a vital fluctuation graph display, or a risk group display described later, to cause user 61 to handle an anomaly in a physical condition of subject 50.

### [1.2.2 Information Recorder 12]

Information recorder 12 records information transmitted and received by transceiver 11. Information recorder 12 is a recording medium capable of recording information and includes, for example, rewritable, nonvolatile memory such as a hard disk drive and a solid state drive. Note that information recorder 12 may record a plurality of features calculated by feature calculator 13.

### [1.2.3 Feature Calculator 13]

Feature calculator 13 includes a computer including, for example, memory and a processor (a microprocessor), and achieves the function of calculating a plurality of features by means of the processor executing a control program stored in the memory. Feature calculator 13 calculates a plurality of features, based on the activity data that is obtained by transceiver 11 and includes the respiratory rate and the heart rate of subject 50. For example, feature calculator 13 obtains sensor data for a time period including target dates and times of detection of a physical condition from activity data obtained by transceiver 11 or recorded on information recorder 12 and calculates hourly features on an hourly basis for each type of sensor data such as the respiratory rate. Here, feature calculator 13 calculates, as a plurality of hourly features, at least a mean value and a maximum value of respiratory rates of subject 50 and a mean value and a maximum value of heart rates of subject 50.

In the present embodiment, feature calculator 13 calculates the following as the plurality of features, based on at least the respiratory rate and the heart rate. That is, feature calculator 13 calculates, among a mean value, a maximum value, a standard deviation, a skewness, a kurtosis, and an impulse factor of each of the respiratory rate, difference data on the respiratory rate, the heart rate, and difference data on the heart rate, at least the mean value and the maximum value of each of the respiratory rate and the heart rate. Here, the impulse factor is obtained by subtracting the mean value from the maximum value. In this manner, feature calculator 13 calculates the plurality of features by performing statistical processing and the like on the activity data.

More specifically, feature calculator 13 calculates, for example, respiratory-rate-related features and heart-rate-related features of subject 50 on an hourly basis.

For example, feature calculator 13 obtains sensor data indicating respiratory rates of subject 50 within a time period including target dates and times of detection of a physical condition from activity data recorded on information recorder 12 or sensor data obtained from sensor 20 and calculates hourly statistical features for the time period.

In more detail, feature calculator 13 obtains, for example, respiratory rate data of a respiratory rate not being zero within a certain hour from the activity data and calculates, as the statistical features, a mean value, a maximum value, a minimum value, a standard deviation, a skewness, a kurtosis, an impulse factor, and the like within the hour from the respiratory rate data obtained. Here, the impulse factor can be calculated from a difference between the maximum value and the mean value (maximum value−mean value) of the respiratory rate data for the hour. Further, feature calculator 13 calculates, as the statistical features, a mean value, a maximum value, a minimum value, a standard deviation, a skewness, a kurtosis, an impulse factor, and the like within the hour from difference data on the respiratory rate data obtained. The difference data on the respiratory rate data obtained is data indicating, for example, a difference between a respiratory rate at time point t and a respiratory rate at a time point t+1 that is one second after time point t, that is, a second-by-second difference of the respiratory rate data. Note that it suffices if feature calculator 13 calculates, as the statistical features, at least a mean value and a maximum value within the hour from the respiratory rate data obtained.

For example, feature calculator 13 also obtains heart rate data indicating heart rates of subject 50 within a time period including target dates and times of detection of a physical condition from activity data recorded on information recorder 12 or sensor data obtained from sensor 20 and calculates hourly statistical features for the time period.

Here, feature calculator 13 obtains, for example, heart rate data of a heart rate not being zero within a certain hour from the activity data and calculates, as the statistical features, a mean value, a maximum value, a minimum value, a standard deviation, a skewness, a kurtosis, an impulse factor, and the like within the hour from the heart rate data obtained. Further, feature calculator 13 calculates, as the statistical features, a mean value, a maximum value, a minimum value, a standard deviation, a skewness, a kurtosis, an impulse factor, and the like within the hour from difference data on the heart rate data obtained. As with the difference data on the respiratory rate data, the difference data on the heart rate data obtained is data indicating, for example, a difference between a heart rate at time point t and a heart rate at a time point t+1 that is one second after time point t, that is, a second-by-second difference of the heart rate data. Note that it suffices if feature calculator 13 calculates, as the statistical features, at least a mean value and a maximum value within the hour from the heart rate data obtained.

Note that feature calculator 13 may calculate food intake or an out-of-bed rate of subject 50 as one of the plurality of features.

That is, for example, feature calculator 13 may calculate a food intake of subject 50 as one of the plurality of features, from recorded data 25 included in activity data. In this case, it suffices if feature calculator 13 calculates a total amount of food intakes in one day in the past from recorded data 25, and then calculates a total sum of food intakes within a time period including target dates and times of detection of a physical condition. Here, in the case where the target dates and times are in time periods in the morning, afternoon, or night, it suffices if feature calculator 13 calculates, for example, a total sum of food intakes during a period from the morning of a previous day of the target date of detection of a physical condition to the morning of the target date, a period from the afternoon of the previous day to the afternoon of the target date, and a period from the night of the previous day to the night of the target date.

For example, feature calculator 13 may also calculate an out-of-bed rate as one of the plurality of features, from activity data obtained by transceiver 11 and recorded on information recorder 12. In this case, it suffices if feature calculator 13 obtains in-or-out-of-bed data indicating whether subject 50 is in or out of bed within a time period including target dates and times of detection of a physical condition from activity data recorded on information recorder 12 or sensor data obtained from sensor 20, and calculates an hourly out-of-bed rate for the time period. In more detail, feature calculator 13 can calculate an out-of-bed rate within a certain hour by, for example, counting up the number of values 1 indicating being out of bed within the certain hour and dividing the number by a total number within the hour (i.e., a total of the number of values 1 indicating being out of bed and the number of values 0 indicating being in bed within the hour).

### [1.2.4 Model Creator 14]

Model creator 14 creates a model that has learned normality or anomaly in an activity data group including a plurality of features. More specifically, model creator 14 creates a model that has learned normality or anomaly in an activity data group including a plurality of features through unsupervised learning using the activity data group.

In the present embodiment, model creator 14 includes a computer including, for example, memory and a processor (a microprocessor), and achieves various functions by means of the processor executing a control program stored in the memory. Model creator 14 obtains activity data in a training time period from activity data recorded on information recorder 12 or sensor data obtained from sensor 20. Note that model creator 14 may obtain recorded data 25 in the training time period and add recorded data 25 in the training time period to the activity data in the training time period.

Model creator 14 also causes feature calculator 13 to calculate hourly features based on the activity data in the training time period. Using the hourly features in the training time period, model creator 14 performs unsupervised learning on a model, thus creating a model that has learned normality or anomaly in an activity data group. Here, the model that has learned normality or anomaly is a model that separates an outlier, based on a decision tree, and is, for example, an isolation forest model. Note that, model creator 14 may perform unsupervised learning by k-means clustering (k-means) on a model using the hourly features in the training time period, thus creating a model that has learned normality or anomaly in an activity data group.

The case where, for example, an isolation forest model or the like is created as the model according to the present embodiment will be described below in detail.

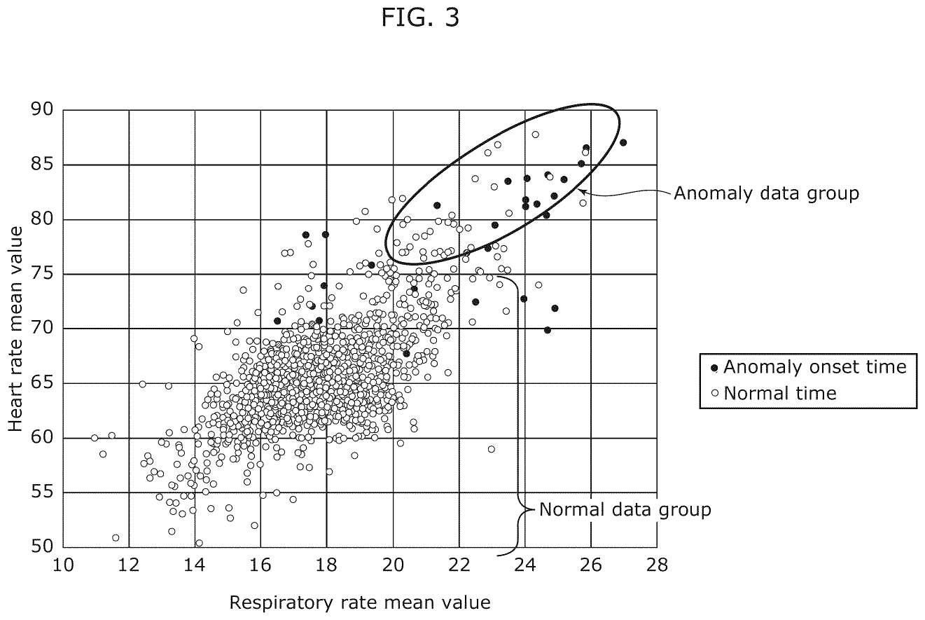

FIG. 3 is a diagram illustrating an example of distributions of an anomaly data group and a normal data group in an activity data group according to the present embodiment. In FIG. 3, its vertical axis indicates the mean value of heart rates, and its horizontal axis indicates the mean value of respiratory rates. The activity data group includes a plurality of types of sensor data such as heart rates and respiratory rates, and includes anomaly data items and normal data items that are intermixed.

FIG. 3 shows that the normal data items outnumber the anomaly data items, and that a distribution of the normal data items is a distribution being dense to some extent, that is, the distribution of the normal data items is not scattered but concentrated. In contrast, many of the anomaly data items are included in a region denoted as anomaly data group indicated in FIG. 3. This shows that the number of the anomaly data items is smaller in number than the number of the normal data items, and that the anomaly data items are at positions different from a distribution position at which the normal data items are concentrated.

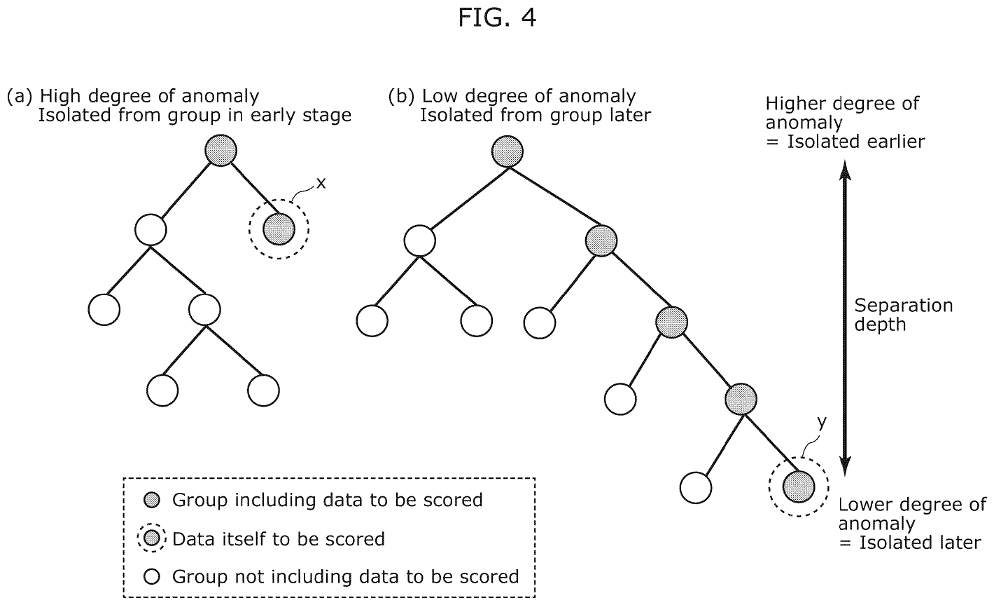

FIG. 4 is a diagram schematically illustrating the model according to the present embodiment. The model illustrated in FIG. 4 is an isolation forest model.

Using hourly features in a training time period, that is, an activity data group in the training time period, model creator 14 creates a model that separates the activity data group on the premise that the anomaly data less frequently occurs than the normal data, and that the anomaly data is different from the normal data in distribution position. More specifically, model creator 14 repeats separation while randomly selecting feature and a threshold value, thus forming a plurality of decision trees. Model creator 14 creates the decision trees in such a manner as to separate an outlier from the other values, thus creating the model that separates the activity data group. In this manner, model creator 14 can create the model that separates, for example, an anomaly data item included in the anomaly data group illustrated in FIG. 3 in an early stage in the decision tree because such an anomaly data item is away from the distribution of the normal data items. Accordingly, the model created by model creator 14 can calculate in which stage in a decision tree a data item is separated (a distance from a root node) as an anomaly score. The model calculates a higher anomaly score for a data item that is separated in an earlier stage in the decision tree (i.e., for a shorter distance of a separated node from the root node). Note that the model includes the plurality of decision trees created. Therefore, an anomaly score output finally is a mean value of anomaly scores obtained from separation depths that are calculated based on a manner of separation in the plurality of decision trees.

For example, as illustrated in (a) of FIG. 4, the model created by model creator 14 isolates (separates) an anomaly data item having an outlier in an early stage as in the case of a node indicated with ‘x’ in FIG. 4 because such an anomaly data item deviates significantly from values of normal data items in a normal data group. The model therefore calculates a high anomaly score for an anomaly data item having an outlier.

In contrast, as illustrated in (b) of FIG. 4, the model created by model creator 14 has difficulty in insolating (separating) a normal data item as in the case of a node indicated with ‘y’ in FIG. 4 because such a normal data item is in a normal data group. The model therefore calculates a low anomaly score for a normal data item.

### [1.2.5 Model Updater 15]

Model updater 15 regularly updates the model created by model creator 14. Model updater 15 regularly updates the model using the activity data obtained by transceiver 11 after the model is created.

Model updater 15 may update the model as frequently as, for example, about every two weeks or about every month. Model updater 15 may update the model as frequently as, for example, every two weeks during a certain time period after the model is created by model creator 14 and may update the model as frequently as, for example, every month after the certain time period.

In the present embodiment, model updater 15 includes a computer including, for example, memory and a processor (a microprocessor), and achieves the model updating function by means of the processor executing a control program stored in the memory. Model updater 15 updates the model by updating structures or conditions of the plurality of decision trees using activity data that is obtained by transceiver 11 after the model has been created. Accordingly, the model can be repeatedly updated with activity data being the activity data accumulated after the creation of the model and added to the activity data used in the creation of the model.

### [1.2.6 Physical Condition Detector 16]

Physical condition detector 16 is implemented using a computer including, for example, a processor (a microprocessor), memory, a communication interface, and the like, and achieves various functions by means of the processor executing a control program stored in the memory. Physical condition detector 16 detects an anomaly in health of subject 50, using the model created by model creator 14 and the plurality of features calculated by feature calculator 13.

As illustrated in FIG. 2, physical condition detector 16 includes anomaly score calculator 161, calculation result recorder 162, graded score calculator 163, and factor analyzer 164.

### [1.2.6.1 Anomaly Score Calculator 161]

Anomaly score calculator 161 obtains an anomaly score indicating a degree of an anomaly in the physical condition per predetermined time period, by inputting the plurality of features calculated by feature calculator 13 into the model that has learned normality or anomaly in an activity data group.

In the present embodiment, anomaly score calculator 161 inputs a plurality of hourly features on a target date of detection of a physical condition of subject 50 that are calculated by feature calculator 13 into the model created by model creator 14. Anomaly score calculator 161 calculates the separation depths based on, for example, the manner of separation in the plurality of decision trees constituting the model illustrated in FIG. 4 and calculates the anomaly score by averaging values of a plurality of the separation depths. Anomaly score calculator 161 records hourly anomaly scores on the target date of detection of the physical condition of subject 50 that are calculated, on calculation result recorder 162.

### [1.2.6.2 Calculation Result Recorder 162]

Calculation result recorder 162 is a recording medium capable of recording a calculation result and includes, for example, rewritable, nonvolatile memory such as a hard disk drive and a solid state drive. In the present embodiment, calculation result recorder 162 records, as a calculation result, an anomaly score calculated by anomaly score calculator 161, a graded score calculated by graded score calculator 163, and the like. Note that calculation result recorder 162 may record, as a calculation result, the factor analyzed by factor analyzer 164.

### [1.2.6.3 Graded Score Calculator 163]

Graded score calculator 163 calculates a graded score for indicating a physical condition anomaly level of subject 50 in a graded manner, based on the anomaly score calculated by anomaly score calculator 161.

In the present embodiment, graded score calculator 163 calculates a daily anomaly-score mean value from hourly anomaly scores of a target date of detection of a physical condition that are recorded on calculation result recorder 162 or calculated by anomaly score calculator 161. Graded score calculator 163 similarly calculates daily anomaly-score mean values of a previous day of the target date of detection of the physical condition and the day before the previous day from hourly anomaly scores of the previous day of the target date and the day before the previous day recorded on calculation result recorder 162. Graded score calculator 163 totalizes the daily anomaly-score means of the target date, the previous day, and the day before the previous day, thus calculating a three-day total score. Note that the three-day total score is an example of a calculation method for calculating a graded score with high precision, and the calculation method is not limited to this. It suffices if graded score calculator 163 performs the calculation within the range from a one-day total score to a five-day total score.

Graded score calculator 163 calculates threshold values for graded scores (will be also referred to as graded threshold values) from a three-day total score group for about 90 days in the past from the target date recorded on calculation result recorder 162. More specifically, graded score calculator 163 calculates the graded threshold values by calculating a mean and a standard deviation of the three-day total score group for the about 90 days in the past.

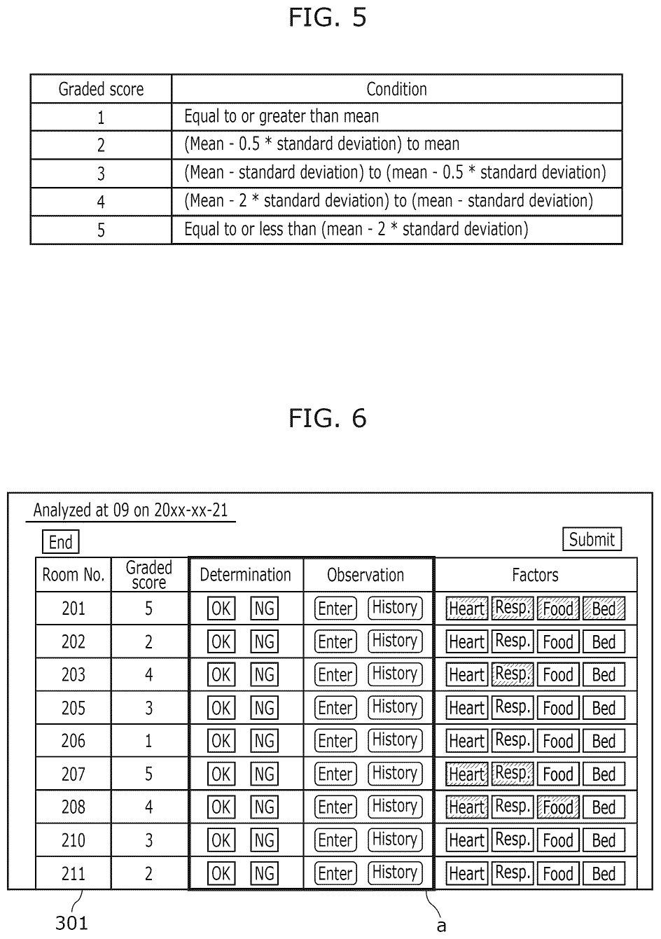

FIG. 5 is a diagram showing an example of graded scores in five levels and conditions for the graded scores according to the embodiment.

When calculating the graded scores in five levels shown in FIG. 5, graded score calculator 163 can calculate threshold values from a mean and a standard deviation as shown in FIG. 5. From the conditions shown in FIG. 5, for example, the threshold value for a graded score of 1 is equal to or greater than the mean, and the threshold value for a graded score of 2 is between the mean and a value calculated by subtraction of a value being half the standard deviation from the mean.

Graded score calculator 163 then applies the threshold values calculated in this manner to the three-day total score of the target date, thus calculating a graded score. More specifically, graded score calculator 163 calculates a value of the graded score by performing, on the three-day total score of the target date, determination using the threshold value calculated under the conditions shown in FIG. 5.

Graded score calculator 163 outputs the value of the graded score calculated to calculation result recorder 162. Graded score calculator 163 may further output the graded score calculated to display terminal 30 via communication network 40 when the value of the graded score calculated is from 1 to 3.

Although FIG. 5 shows an example of the case where graded score calculator 163 calculates the graded scores in five levels, this is not limiting. Graded score calculator 163 may calculate graded scores in two to four levels.

Here, with reference to FIG. 5, an example of a method for calculating the threshold values for the graded scores will be described.

### [1.2.6.4 Factor Analyzer 164]

Factor analyzer 164 performs factor analysis when the graded score is greater than or equal to a predetermined value to analyze, for each of elements included in the activity data, whether the element is a factor for the graded score being greater than or equal to the predetermined value. Here, the elements include, for example, food intake, the respiratory rate, the heart rate, or an out-of-bed rate of subject 50 during the predetermined time period. In addition, the predetermined value is a value at which handling a sign of an anomaly in health is needed. For example, in the case where the graded scores are in five levels, the predetermined value may be determined to be four or five, in the case where the graded scores are in three levels, the predetermined value may be determined to be three, and in the case where the graded scores are in two levels, the predetermined value may be determined to be two.

In the present embodiment, in the case where the graded score calculated by graded score calculator 163 is four or five, factor analyzer 164 performs a factor analysis on elements including heart rates, respiratory rates, out-of-bed rates, and food intakes, which are included in activity data used to calculate features. In the case where the activity data used to calculate features includes only the heart rates and the respiratory rates, it suffices if the factor analysis is performed on elements including the heart rates and the respiratory rates.

For example, factor analyzer 164 converts a plurality of features of each element in an entire time period used to calculate a graded score into data of a plurality of daily features of the element to calculate mean values and standard deviations in the entire time period used to calculate the graded score. In the present embodiment, factor analyzer 164 converts a plurality of features of each element in three days into data of a plurality of daily features of the element to calculate mean values and standard deviations of the element in the three days.

Factor analyzer 164 then makes an analysis showing that the element does not form a factor when Expression 1 shown below is established, and makes an analysis showing that the element forms a factor when Expression 1 shown below is not established.

(Mean value−2*standard deviation)≤(feature of the element at target date and time)≤(mean value+2*standard deviation) (Expression 1)

Note that Expression 1 is the use of such a nature of standard deviation that 95.45% of all data items are distributed within the range that is twice as much as mean value±standard deviation.

Factor analyzer 164 outputs the graded score and the element analyzed to be a factor by the factor analysis to calculation result recorder 162. Factor analyzer 164 may also output the graded score and the element analyzed to be a factor by the factor analysis to display terminal 30 via communication network 40.

### [1.3 Display Terminal 30]

Display terminal 30 is implemented using a computer including, for example, a processor (a microprocessor), memory, a communication interface, a user interface, and the like. Display terminal 30 is a terminal possessed by user 61 such as the monitoring person who monitors subject 50, and is, for example, a tablet or a smartphone. Display terminal 30 may be a mobile computer or a desktop computer connected to a display device.

In the present embodiment, display terminal 30 can be checked by user 61 such as the monitoring person who monitors subject 50. Display terminal 30 is connected to communication network 40, and when, for example, a graded score is obtained from information management server 10, causes the user interface to present a display for user 61 to handle an anomaly in the physical condition of subject 50. The user interface can cause a display device to present a display according to, for example, an input from user 61.

FIG. 6 to FIG. 8 are diagrams each illustrating an example of a display for handing an anomaly in a physical condition of subject 50 according to the present embodiment.

FIG. 6 illustrates an example of graded score display screen 301 according to the embodiment. More specifically, FIG. 6 illustrates graded score display screen 301, which is an example of an application screen to be seen by user 60 or user 61 being an on-site staff member and displays a graded score and the like of subject 50 for early finding and handling an anomaly in health of subject 50. Graded score display screen 301 is displayed on display terminal 30 by a touch or the like to make a selection on a menu screen that is displayed when an application starts on display terminal 30. In FIG. 6, one subject 50 is resident at each of rooms numbered 201 to 211, and graded scores of respective subjects 50 are indicated. FIG. 6 illustrates that, in the case where a graded score is four or five, one or more factors of the graded score are indicated as one or more of the elements including heart rate, respiratory rate, food intake, and out-of-bed rate, and the one or more elements being the one or more factors are hatched. A region indicated by ‘a’ in FIG. 6 illustrates an input field in which user 60 being an on-site staff member such as a nurse is to make an input. In this input field, validities and the like of values of graded scores are to be input.

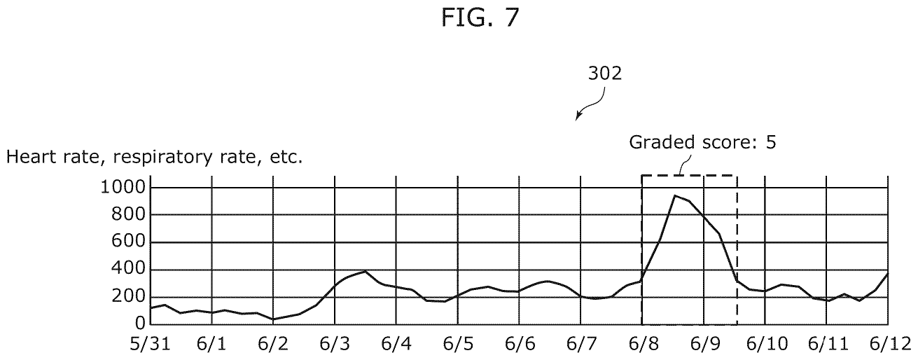

FIG. 7 illustrates an example of vital fluctuation graph display screen 302 according to the embodiment. More specifically, FIG. 7 illustrates vital fluctuation graph display screen 302, which is another example of the application screen to be seen by user 60 or user 61 being an on-site staff member and displays a vital fluctuation graph and the like of a specific subject 50 for early finding and handling an anomaly in health of subject 50. Vital fluctuation graph display screen 302 is also displayed on display terminal 30 by a touch or the like to make a selection on a menu screen that is displayed when an application starts on display terminal 30. FIG. 7 illustrates a range of vital information in the vital fluctuation graph of the specific subject 50 in which a graded score indicates, for example, five. Accordingly, a cause of the graded score of the specific subject 50 indicating, for example, five can be ascertained early.

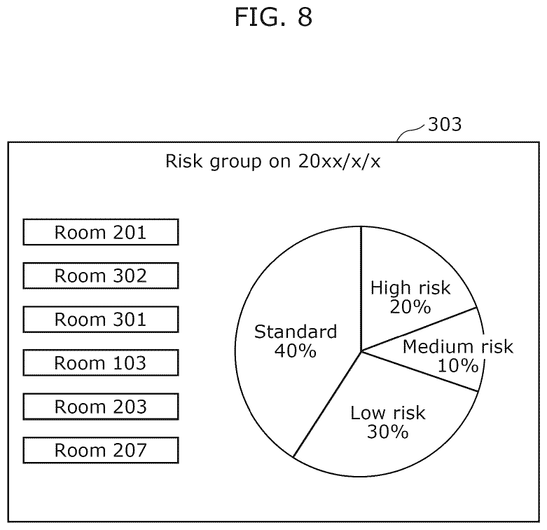

FIG. 8 illustrates an example of risk group display screen 303 according to the embodiment. More specifically, FIG. 8 illustrates risk group display screen 303, which is still another example of the application screen to be seen by user 60 or user 61 being an on-site staff member and displays risk groups of the entire facility in which subject 50 is resident, for early finding and handling an anomaly in health of subject 50. Risk group display screen 303 is also displayed on display terminal 30 by a touch or the like to make a selection on a menu screen that is displayed when an application starts on display terminal 30. FIG. 8 illustrates, in the form of a pie chart, a risk distribution including proportions of the risk groups in the entire facility consisting of a plurality of rooms such as room 201. The example illustrated in FIG. 8 shows that high risk indicating a graded score of five is 20%, medium risk indicating a graded score of four is 10%, low risk indicating a graded score of 3 is 30%, and standard (no risk) indicating a graded score of one or two is 40%. In this manner, the risk distribution of the entire facility is visualized and thus can be summarized. This enables, for example, assignment of an experienced, expert nurse to a room indicated as the medium risk and assignment of a more experienced, expert nurse to a room indicated as the high risk. This similarly enables assignment of an experienced carer to a room indicated as the low risk and assignment of a carer to a room indicated as the standard. In short, since the risk distribution of the entire facility is visualized and can be summarized, right on-site staff members limited in number can be assigned to right places in the entire facility. Accordingly, handling of an anomaly in a physical condition or a sign of an anomaly in a physical condition of subject 50 can be performed more promptly and more appropriately.

### [2 Operation of Information Management Server 10]

Next, operation of information management server 10 configured in the above-described manner will be described.

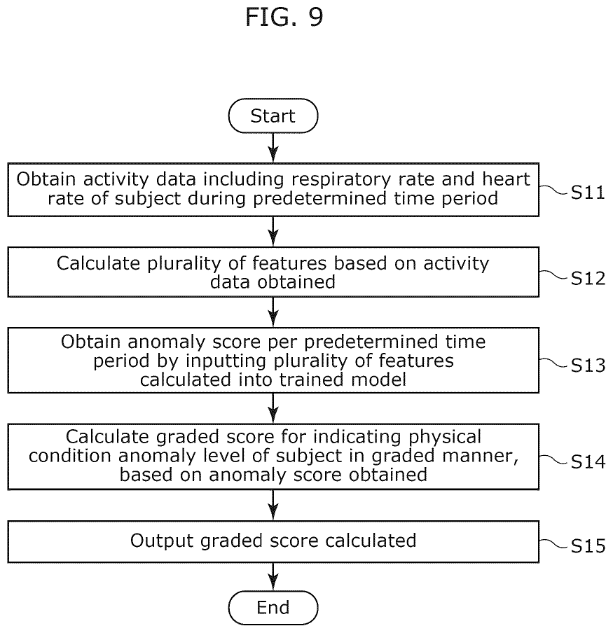

FIG. 9 is a flowchart illustrating an outline of operation of a physical condition detection device according to the present embodiment. An example of the physical condition detection device according to the present embodiment is information management server 10. The physical condition detection device is only required to include at least transceiver 11, feature calculator 13, anomaly score calculator 161, and graded score calculator 163 in the configuration of information management server 10 described above.

First, in the physical condition detection device according to the present embodiment, transceiver 11 obtains activity data including the respiratory rate and the heart rate of subject 50 during a predetermined time period (S11). Next, feature calculator 13 calculates a plurality of features, based on the activity data obtained in step S11 (S12). Next, anomaly score calculator 161 obtains an anomaly score per predetermined time period by inputting the plurality of features calculated in step S12 into a model that has learned in advance normality or anomaly in an activity data group (S13). Next, graded score calculator 163 calculates a graded score for indicating a physical condition anomaly level of subject 50 in a graded manner, based on the anomaly score obtained in step S13 (S14). Graded score calculator 163 then outputs the graded score calculated in step S14 (S15).

Subsequently, as an example of the operation (an operation example) of the physical condition detection device described with reference to FIG. 9, an operation example of information management server 10 according to the present embodiment will be described.

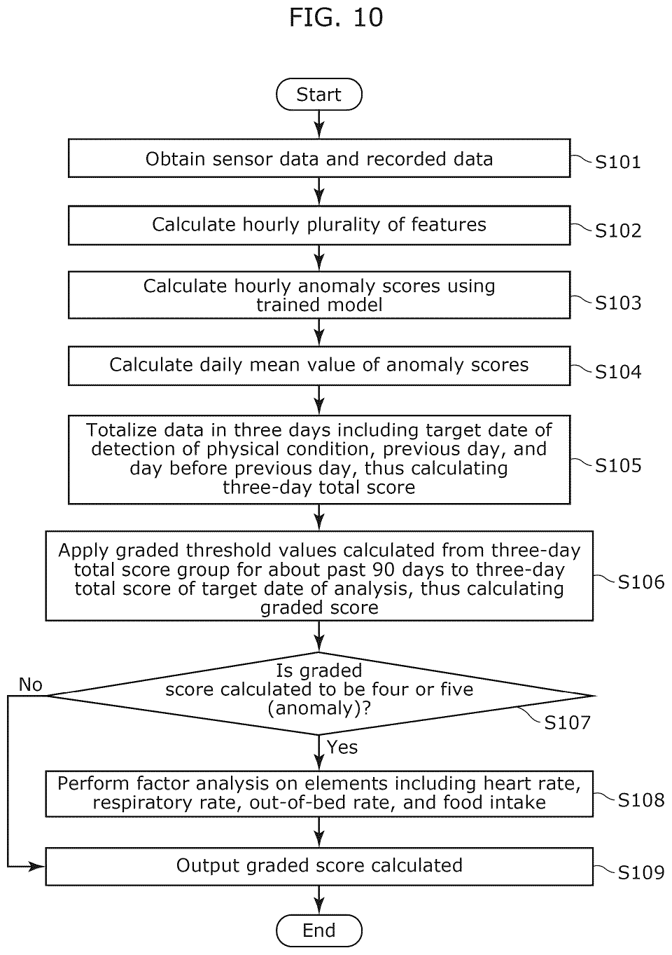

FIG. 10 is a flowchart illustrating the operation example of information management server 10 according to the present embodiment.

In information management server 10, first, transceiver 11 obtains sensor data and recorded data 25 (S101). In the present embodiment, transceiver 11 obtains activity data that includes at least respiratory rates and heart rates of subject 50 in a predetermined time period.

Next, feature calculator 13 calculates a plurality of hourly features from the sensor data and recorded data 25 obtained in step S101 (S102). In the present embodiment, feature calculator 13 calculates a plurality of hourly features on a target date of detection of a physical condition of subject 50 based on the activity data including at least respiratory rates and heart rates of subject 50 obtained by transceiver 11.

Next, physical condition detector 16 calculates hourly anomaly scores using a pre-trained model, from the plurality of hourly features calculated in step S102 (S103). In the present embodiment, anomaly score calculator 161 inputs the plurality of features calculated by feature calculator 13 into a model created by model creator 14, and thereby obtains anomaly scores each indicating a degree of an anomaly in a physical condition per hour in a predetermined time period including the target date.

Next, physical condition detector 16 calculates a daily mean value of anomaly scores from the hourly anomaly scores calculated in step S103 (S104). In the present embodiment, graded score calculator 163 calculates anomaly-score daily mean values from the hourly anomaly scores in the predetermined time period including the target date of detection of a physical condition of subject 50.

Next, physical condition detector 16 totalizes data in three days including the target date of detection of the physical condition of subject 50, its previous day, and the day before the previous day, thus calculating a three-day total score (S105). In the present embodiment, graded score calculator 163 calculates the three-day total score by calculating daily anomaly-score mean values of a previous day of the target date and the day before the previous day and totalizing daily anomaly-score means of the target date, the previous day, and the day before the previous day.

Next, physical condition detector 16 applies graded threshold values calculated from a three-day total score group for about 90 days in the past to the three-day total score of the target date of detection of the physical condition calculated in step S105, thus calculating the graded score (S106). In the present embodiment, graded score calculator 163 calculates the graded threshold value by calculating a mean and a standard deviation of the three-day total score group for the about 90 days in the past from the target date. Graded score calculator 163 then applies the graded threshold values calculated to the three-day total score of the target date, thus calculating the graded score. Note that the graded score here indicates any one of values in five levels from one to five.

Next, physical condition detector 16 checks whether the graded score calculated in step S106 indicates a value of four or five, that is, a value indicating an anomaly (S107).

When the graded score takes a value of four or five in step S107 (Yes in S107), physical condition detector 16 performs factor analysis about elements including food intake, respiratory rate, heart rate, and out-of-bed rate (S108). In the present embodiment, in the case where the graded score calculated is four or five, factor analyzer 164 performs a factor analysis on elements including heart rates, respiratory rates, out-of-bed rates, and food intakes, which are included in activity data used to calculate features.

Next, physical condition detector 16 outputs the graded score and an element which has been analyzed to be a factor by the factor analysis (S109). In the present embodiment, factor analyzer 164 outputs the graded score and the element analyzed to be a factor by the factor analysis.

When the graded score does not take a value of four or five in step S107 (No in S107), physical condition detector 16 outputs the graded score in step S109.

### [3 Advantageous Effects etc.]

As described above, the physical condition detection device and the like according to the present embodiment is capable of detecting a sign of an anomaly in health of subject 50, that is, a small change in the physical condition that may lead to an anomaly in health of subject 50. More specifically, the physical condition detection device and the like according to the present embodiment calculates a plurality of features from activity data and inputs the plurality of features calculated into a model that is capable of detecting a sign of an anomaly in health by learning normality or anomaly in an activity data group. The physical condition detection device and the like according to the present embodiment calculates, based on the anomaly score obtained as a result of inputting the plurality of features calculated into the model, a graded score indicating evaluation of a physical condition anomaly level of subject 50 in a graded manner.

Accordingly, it is possible to become aware of (detect) a sign of an anomaly in health of subject 50 from the graded score. Therefore, it is possible to detect a sign of an anomaly in health of subject 50 that even a health care worker providing care or nursing to subject 50 fails to find.

Furthermore, since the graded score makes it easy for an on-site staff member such as a health care worker providing care or nursing to subject 50 to grasp a degree of the anomaly, handling of various small changes in the physical condition of subject 50, that is, various signs of an anomaly in health that may lead to an anomaly in health in daily life of subject 50, is facilitated.

When the graded score is greater than or equal to a predetermined value indicating the need of handling a sign of an anomaly in health, the physical condition detection device and the like according to the present embodiment may perform factor analysis to analyze, for each of elements included in the activity data, whether the element is a factor for the graded score being greater than or equal to the predetermined value. Further, when the graded score is greater than or equal to the predetermined value indicating the need of handling a sign of an anomaly in health, whether a factor for the small change in the physical condition leading to the anomaly in health lies in, for example, the heart rate or the respiratory rate, and the like are notified together.

Accordingly, a health care worker who provides care or nursing to subject 50 can handle the sign of the anomaly in health early and appropriately with the notified factor as a clue.

Here, the activity data is obtained on-site day-by-day and includes at least the respiratory rate and the heart rate among food intake, the respiratory rate, the heart rate, and an out-of-bed rate of subject 50 during a predetermined period. Accordingly, the plurality of features are calculated from activity data items obtained on-site day-by-day, and thus a sign of an anomaly in health can be detected with higher precision.

The plurality of features according to the present embodiment are, for example, a mean value, a maximum value, a standard deviation, a skewness, a kurtosis, and an impulse factor of each of the respiratory rate of subject 50, difference data on the respiratory rate, the heart rate of subject 50, and difference data on the heart rate. Here, the impulse factor is obtained by subtracting the mean value from the maximum value. In the present embodiment, at least the mean value and the maximum value of each of the respiratory rate and the heart rate of subject 50 are calculated as the plurality of features. In this manner, the physical condition detection device and the like according to the present embodiment calculates the plurality of features by performing statistical processing and the like on the activity data. Accordingly, from the plurality of features calculated, an anomaly score can be obtained with higher precision using a trained model.

The model trained in the present embodiment (also referred as a trained model) is a model that has learned normality or anomaly in the activity data group through unsupervised learning using the activity data group. That is to say, in the present embodiment, a trained model capable of detecting a sign of an anomaly in health is created by performing the unsupervised learning on the model using the activity data items obtained on-site day-by-day. Accordingly, by performing unsupervised learning using activity data of subject 50, it is possible to create a trained model that is capable of detecting a sign of an anomaly in health, without a burden on on-site staff members such as a health care worker for subject 50.

Here, innovations made in the creation of the model according to the present embodiment will be described.

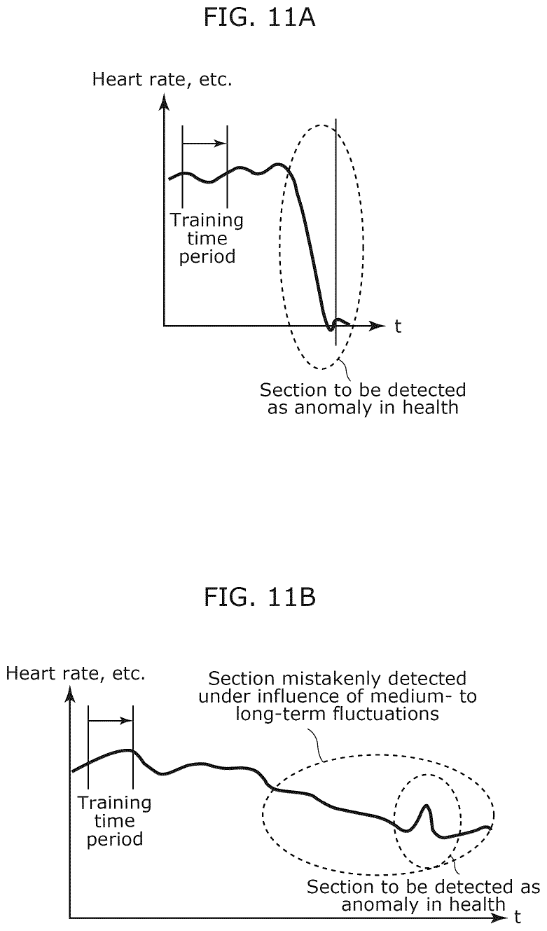

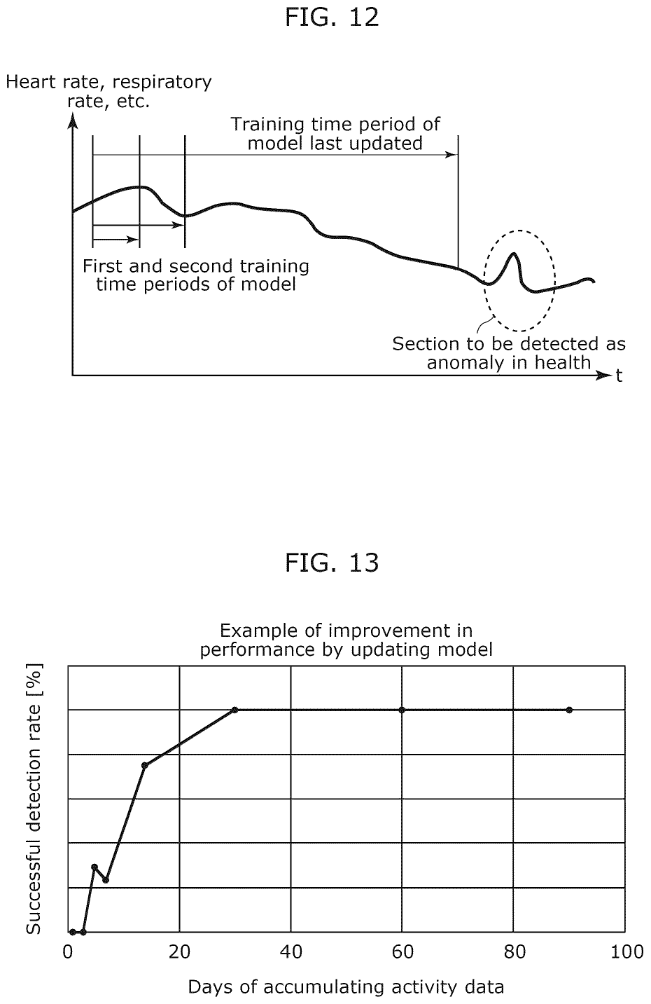

FIG. 11A and FIG. 11B are diagrams for schematically describing anomaly detection in the case of long-term operation using a model according to a comparative example. FIG. 12 is a diagram for schematically describing anomaly detection in the case of long-term operation using a model according to the present embodiment. In each of FIG. 11A to FIG. 12, its horizontal axis indicates time, and its vertical axis indicates activity data such as heart rate. FIG. 11A to FIG. 12 each schematically illustrate a time period of the activity data used for training the model and a section that is to be detected as an anomaly in health of subject 50.

The model according to the comparative example is, for example, the model according to PTL 1 and is one model created by training using activity data in a short training time period.

Therefore, for example, in the case where an interval between the training time period and the section to be detected as an anomaly in health is relatively short as illustrated in FIG. 11A, the anomaly in health can be detected using the model according to the comparative example. In contrast, for example, in the case where an interval between the training time period and the section to be detected as an anomaly in health is relatively long as illustrated in FIG. 11B, there is an influence of medium- to long-term fluctuations in activity data of subject 50, and even the model according to the comparative example may mistakenly detect an anomaly in health. This is because a disease of subject 50 causes medium- to long-term fluctuation of the activity data of subject 50 because of seasonal and environmental influences. In short, since the model according to the comparative example is created by training using activity data in an early, relatively short training time period, the comparative example is highly likely to fail to deal with medium- to long-term fluctuations in activity data of subject 50 in the case of long-term operation. In other words, a small number of activity data items used in the training makes the model according to the comparative example highly likely to fail to detect a plurality of small changes in a physical condition or a complex pattern of small change in a physical condition that may lead to an anomaly in health.

In contrast, the model according to the present embodiment is repeatedly updated with, for example, activity data that is accumulated on a two-week or monthly basis. That is, the model according to the embodiment is repeatedly updated with activity data being the activity data accumulated after the creation of the model and added to the activity data used in the creation of the model.

Therefore, as illustrated in FIG. 12 for example, the model according to the present embodiment has learned or been updated with activity data up to immediately before a section to be detected as an anomaly in health, thus being capable of detecting an anomaly in health in the section to be detected as an anomaly in health. In short, the model according to the present embodiment is capable of detecting a sign of an anomaly in health while dealing with fluctuations due to a medium- to long-term disease of subject 50 or medium- to long-term fluctuations due to environmental influences.

FIG. 13 is a graph schematically illustrating improvement in performance by updating the model according to the present embodiment.

In FIG. 13, its horizontal axis indicates successful detection rate of anomaly in health, and its vertical axis indicates the number of days of accumulating activity data. FIG. 13 shows that the successful detection rate improves with an increase in the number of days of accumulating activity data that the model learns and is updated with, and plateaus out at a certain number of activity data items.

In short, it is understood that, in the case of medium- to long-term operation, the model according to the present embodiment improves in detection performance with an increase in the activity data accumulated.

The model according to the comparative example, namely, the model according to PTL 1 is created with, as training data, activity data in a time period during which a physical condition of subject 50 is in a normal state. Therefore, it is necessary to make an on-site staff member determine whether the activity data in the time period is activity data indicating only a normal state or activity data including an anomaly state by referring to recorded data 25 about care and the like. This is a burden on the on-site staff member.

In contrast, the model according to the present embodiment is created by performing unsupervised learning using activity data including anomaly data items and normal data items that are intermixed, using such a nature that anomaly data less frequently occurs than normal data, and that the anomaly data is different from the normal data in distribution position. Accordingly, the model can be created with activity data of subject 50 without a burden on an on-site person such as a health care worker for subject 50. Using this nature, the model according to the present embodiment can be created as a model that determines how far an activity data item being a target of detection of a physical condition is away from a part where a distribution of normal data is concentrated. A model that separates an outlier based on a decision tree, such as an isolation forest model, can be created through unsupervised learning using the nature. Therefore, the model can be used as the model according to the present embodiment.

### Working Example 1

In the description of the above embodiment, it is assumed that display terminal 30 performs, for example, displays as illustrated in FIG. 6 to FIG. 8, that is, displays for handling an anomaly in a physical condition of subject 50. However, this is not limiting.

Display terminal 30 may perform a linkage display in which a graded score calculated by physical condition detector 16 is linked to recorded data 25 including recorded details of nursing or care for subject 50.

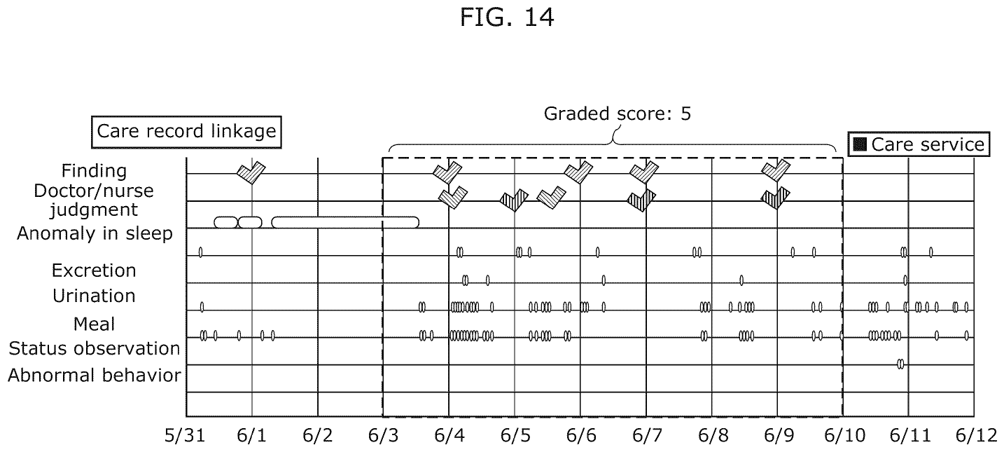

FIG. 14 is a diagram illustrating an example of the linkage display displayed by display terminal 30 according to Working Example 1.

FIG. 14 illustrates an example of the linkage display in which a care record including recorded details of care for subject 50 and graded scores are displayed in a linked manner. A screen of the linkage display illustrated in FIG. 14 is a screen that an on-site staff member performing a care service can check.

In the linkage display illustrated in FIG. 14, there are check marks at points on the care record at each which an on-site staff member makes a finding, or a judgment is made by a doctor or a nurse. In the linkage display illustrated in FIG. 14, a dotted frame indicating a time period during which the graded score shows five, that is, the predetermined value indicating the need of handling an anomaly in a physical condition of subject 50, is superimposed on the care record. In the linkage display illustrated in FIG. 14, dates and times of anomalies in sleep, dates and times of excretion and urination, dates and times of meals, and the like are also displayed.

An on-site staff member who watches the linkage display illustrated in FIG. 14 can ascertain a cause of a sign of an anomaly in health of subject 50 promptly from living conditions of subject 50 when the graded score shows five.

### Working Example 2

In Working Example 2, an incident finding case in which a sign of an anomaly in health of subject 50, that is, a small change in a physical condition that may lead to the anomaly in health of the subject is successfully detected will be described.

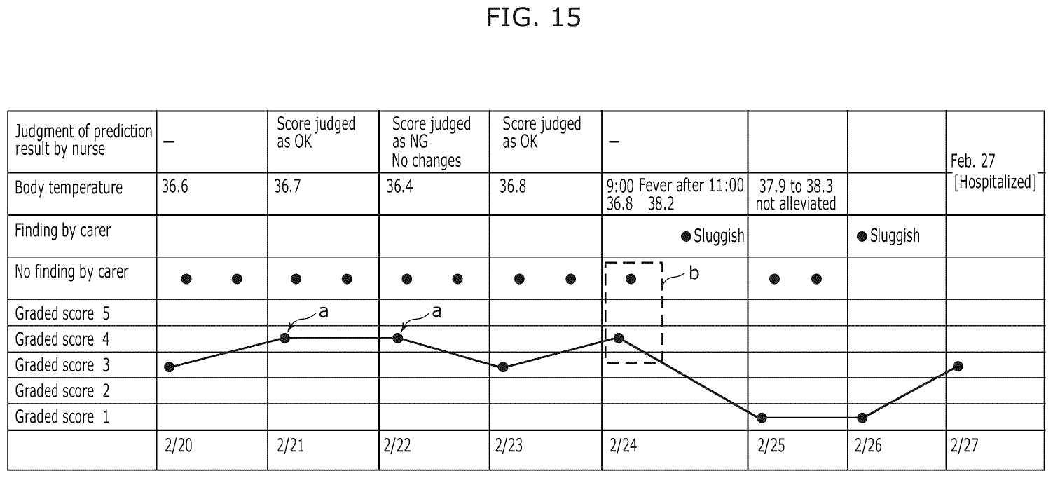

FIG. 15 is a diagram illustrating incident finding case 1 according to Working Example 2. FIG. 15 illustrates an example of the case where subject 50 is hospitalized for suspected pneumonia with a high sudden-onset factor, on February 27.

In FIG. 15, a result of calculating a graded score is present at 8:00 every morning, and whether there is a finding about a physical condition of subject 50 by a carer is input at 10:00 or 15:00 every day. When the graded score is four or greater, a nurse checks a condition of subject 50 after 18:00 and inputs whether the graded score is correctly presented, in the form of OK or NG.

As illustrated in FIG. 15, on February 21 and February 22, when graded scores indicated with ‘a’ show four, the nurse judges the graded scores as OK, and on February 24, when a graded score indicated with ‘b’ shows four, there is no finding by the carer as of 10:00. In addition, after 11:00 on February 24, subject 50 develops a fever and does not alleviate the fever, but there is no finding by the carer (i.e., no problem) on February 25. However, on February 27, subject 50 is hospitalized for suspected pneumonia with a high sudden-onset factor.

In contrast, the result of calculating the graded score shows that signs of an anomaly in health of the subject, that is, small changes in the physical condition that may lead to the anomaly in health of the subject are detected on February 21 and February 22, when the graded scores indicated with ‘a’ show four, and on February 24, when the graded score indicated with ‘b’ shows four.

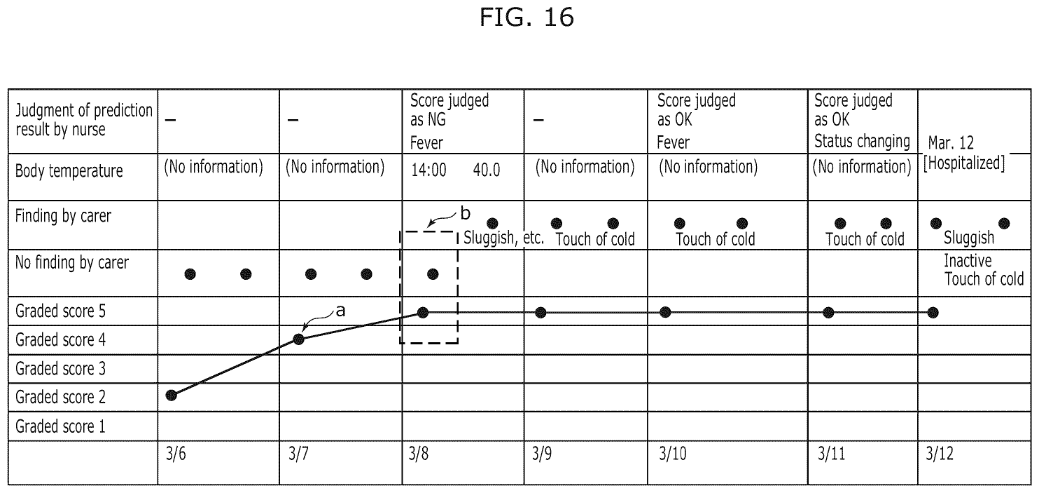

FIG. 16 is a diagram illustrating incident finding case 2 according to Working Example 2. FIG. 16 illustrates an example of the case where another subject 50 is hospitalized due to development of a fever on March 12. Judgments by a nurse, findings by a carer, and the like in FIG. 16 are similar to those in FIG. 15, and thus description thereof will be omitted.

As illustrated in FIG. 16, on March 7, when a graded score indicated with ‘a’ shows four, there is no finding by the carer (i.e., no problem). In addition, on March 8, when a graded score indicated with ‘a’ shows five, there is no finding by the carer as of 10:00, but there are findings such as looking sluggish and inactive, having a touch of a cold, and being painful as of 15:00. On March 8, when the graded score indicated with ‘a’ shows five, the nurse judges the graded score as OK. Afterward, there are findings by the carer, and subject 50 is sluggish and inactive and has a touch of a cold as of March 12, thus being hospitalized.

In contrast, according to the result of calculating the graded score, on March 7, the graded score shows four as indicated with ‘a’, on March 8, the graded score shows five as indicated with ‘b’, and afterward, the graded score continues to show five. In short, it is understood that a sign of an anomaly in health of subject 50, that is, a small change in a physical condition that may lead to the anomaly in health of subject 50 is detected on March 7, when the graded score shows four.

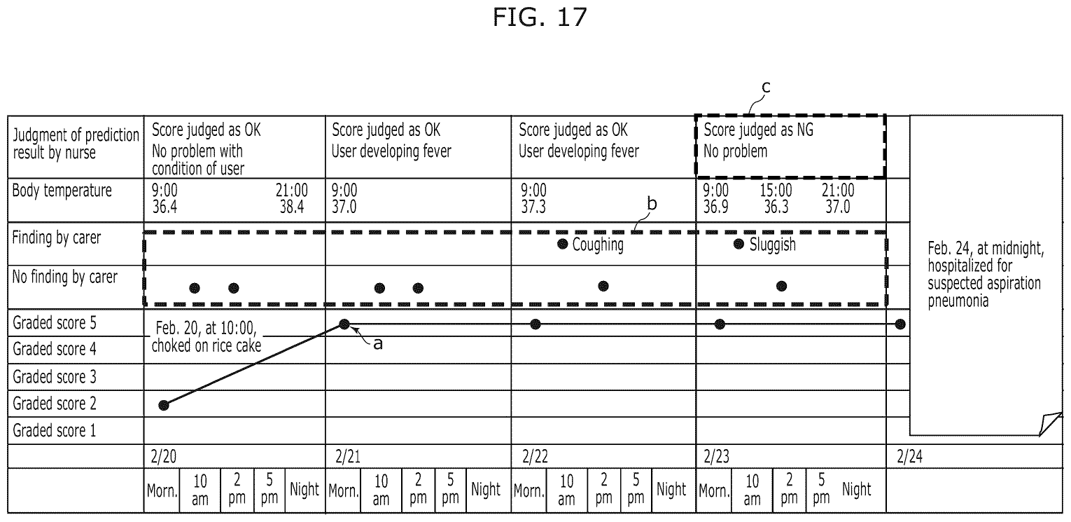

FIG. 17 is a diagram illustrating incident finding case 3 according to Working Example 2. FIG. 17 illustrates an example of the case where yet another subject 50 chokes on a rice cake at 10:00 on February 20 and is hospitalized for suspected aspiration pneumonia at midnight on February 24. Judgments by a nurse, findings by a carer, and the like in FIG. 17 are similar to those in FIG. 15, and thus description thereof will be omitted.

As illustrated in FIG. 17, on February 21, the next day of February 20 when subject 50 chokes on a rice cake, the graded score indicated with ‘a’ shows five and then the graded score continues to show five until the day of the hospitalization. Meanwhile, during a period from February 21 to February 23, the carer inputs findings that subject 50 is coughing, sluggish, and the like but judges that there is no finding (no problem) in some time periods. In addition, on February 23, the nurse judges the graded score showing five as NG and judges that there is no problem with subject 50 although a physical condition of subject 50 has not recovered. That is, the nurse overlooks a sign of anomaly in health of subject 50 on February 23.

In contrast, according to the result of calculating the graded score, the graded score shows five from February 21 as indicated with ‘a’ and afterward, the graded score continues to show five. In short, it is understood that a sign of an anomaly in health of subject 50, that is, a small change in a physical condition that may lead to the anomaly in health of subject 50 is detected since February 21 when the graded score shows five, that is, three days prior to February 24 when subject 50 is hospitalized.

### (Possibilities for Other Aspects)

Hereinbefore, information management server 10 and the like according to an exemplary embodiment, working examples, etc., that is, a physical condition detection method and a physical condition detection device according to an exemplary embodiment, working examples, etc., have been described, but the present disclosure is not limited to the above exemplary embodiment and working examples.

Note that each of the processing units included in information management server 10 according to the above exemplary embodiment, working examples, etc., is typically implemented as a large-scale integrated (LSI) circuit, which is an integrated circuit (IC). These may take the form of individual chips, or may be partially or entirely packaged into a single chip.

Such IC is not limited to an LSI, and thus may be implemented as a dedicated circuit or a general-purpose processor. Alternatively, a field programmable gate array (FPGA) that allows for programming after the manufacture of an LSI, or a reconfigurable processor that allows for reconfiguration of the connection and the setting of circuit cells inside an LSI may be employed.

The present disclosure may also be implemented as a physical condition detection method executed by information management server 10 and the like, that is, a physical condition detection device.

Moreover, in the above exemplary embodiment, the constituent elements may be configured in the form of a dedicated hardware product or may be implemented by executing a software program suited to such constituent elements. The constituent elements may be implemented by a program executor such as a central processing unit (CPU) or a processor reading out and executing the software program recorded on a recording medium such as a hard disk or semiconductor memory.

Also, the divisions of the functional blocks shown in the block diagrams are mere examples, and thus a plurality of functional blocks may be implemented as a single functional block, or a single functional block may be divided into a plurality of functional blocks, or one or more functions may be moved to another functional block. Also, the functions of a plurality of functional blocks having similar functions may be processed by single hardware or software in a parallelized or time-divided manner.

Also, the processing order of executing the steps shown in the flow charts is a mere illustration for specifically describing the present disclosure, and thus may be an order other than the order described above. Also, one or more of the steps may be executed simultaneously (in parallel) with another step.

A physical condition detection device according to one or more aspects has been described above based on an exemplary embodiment and working examples, but the present disclosure is not limited to the exemplary embodiment and working examples. The one or more aspects may thus include forms achieved by making various modifications to the above embodiment and working examples that can be conceived by those skilled in the art, as well as forms achieved by combining constituent elements in different embodiments, working examples, and variations, without materially departing from the spirit of the present disclosure.

### INDUSTRIAL APPLICABILITY

The present disclosure is applicable to physical condition detection methods, physical condition detection devices, and recording media, and is applicable to a physical condition detection method, a physical condition detection device, and a recording medium capable of detecting, for example, a small change in a physical condition that may lead to an anomaly in health of a subject, as a sign of an anomaly in health of the subject.

1. A physical condition detection method performed by a computer, the method comprising:
obtaining activity data including a respiratory rate and a heart rate of a subject during a predetermined time period;
calculating a plurality of features, based on the activity data obtained;
obtaining an anomaly score indicating a degree of an anomaly in a physical condition per the predetermined time period, by inputting the plurality of features calculated into a model that has learned normality or anomaly in an activity data group including a plurality of features;
calculating a graded score for indicating a physical condition anomaly level of the subject in a graded manner, based on the anomaly score obtained; and
outputting the graded score calculated.

2. The physical condition detection method according to claim 1,
wherein the calculating of the graded score includes:
performing factor analysis when the graded score is greater than or equal to a predetermined value to analyze, for each of elements included in the activity data, whether the element is a factor for the graded score being greater than or equal to the predetermined value, and
the outputting of the graded score includes:
outputting the graded score and an element which has been analyzed to be the factor by the factor analysis.

3. The physical condition detection method according to claim 1,
wherein the activity data includes at least the respiratory rate and the heart rate among food intake, the respiratory rate, the heart rate, and an out-of-bed rate of the subject during the predetermined time period, the out-of-bed rate being a rate at which the subject is out of bed.

4. The physical condition detection method according to claim 3,
wherein in the calculating of the plurality of features,
among a mean value, a maximum value, a standard deviation, a skewness, a kurtosis, and an impulse factor of each of the respiratory rate, difference data on the respiratory rate, the heart rate, and difference data on the heart rate, at least the mean value and the maximum value of each of the respiratory rate and the heart rate are calculated as the plurality of features, the impulse factor being obtained by subtracting the mean value from the maximum value.

5. The physical condition detection method according to claim 1,
wherein the model is a model that has learned normality or anomaly in the activity data group through unsupervised learning using the activity data group.

6. The physical condition detection method according to claim 5,
wherein the model is a model that separates an outlier, based on a decision tree.

7. The physical condition detection method according to claim 6,
wherein the model is an isolation forest model.

8. The physical condition detection method according to claim 5,
wherein the model is regularly updated using the activity data obtained.

9. The physical condition detection method according to claim 5,
wherein the outputting of the graded score includes:
outputting the graded score calculated to a terminal possessed by a monitoring person who monitors the subject; and
causing a user interface of the terminal to present a display for the monitoring person to handle an anomaly in a physical condition of the subject.

10. A physical condition detection device comprising:
a transceiver that obtains activity data including a respiratory rate and a heart rate of a subject during a predetermined time period;
a feature calculator that calculates a plurality of features, based on the activity data obtained;
an anomaly score calculator that obtains an anomaly score indicating a degree of an anomaly in a physical condition per the predetermined time period, by inputting the plurality of features calculated into a model that is created by a model creator and has learned normality or anomaly in an activity data group including a plurality of features; and
a graded score calculator that calculates a graded score for indicating a physical condition anomaly level of the subject in a graded manner, based on the anomaly score obtained,
wherein the transceiver outputs the graded score calculated.

11. A non-transitory computer-readable recording medium having recorded thereon a program for causing a computer to execute:
obtaining activity data including a respiratory rate and a heart rate of a subject during a predetermined time period;
calculating a plurality of features, based on the activity data obtained;
obtaining an anomaly score indicating a degree of an anomaly in a physical condition per the predetermined time period, by inputting the plurality of features calculated into a model that has learned normality or anomaly in an activity data group including a plurality of features;
calculating a graded score for indicating a physical condition anomaly level of the subject in a graded manner, based on the anomaly score obtained; and
outputting the graded score calculated.
